(19) United States
(12) Patent Application Publication
(10) Pub. No.: US 2023/0050625 A1
(43) Pub. Date: Feb. 16, 2023

(54) COIL WIRE FOR NAVIGATION IN VASCULAR TORTUOSITY AND METHODS OF USING THE COIL WIRE

(71) Applicant: THE UAB RESEARCH FOUNDATION, Birmingham, AL (US)

(72) Inventor: Jesse G.A. Jones, Mountain Brook, AL (US)

(21) Appl. No.: 17/972,780

(22) Filed: Oct. 25, 2022

Related U.S. Application Data
(63) Continuation-in-part of application No. PCT/US2021/029700, filed on Apr. 28, 2021.
(60) Provisional application No. 63/016,730, filed on Apr. 28, 2020.
(60) Provisional application No. 63/017,131, filed on Apr. 29, 2020.

(51) Int. Cl. A61B 10/02

(52) U.S. Cl. CPC A61B 10/02

(57) ABSTRACT

A coil wire and method of using the coil wire to navigate blood vessels are provided. The coil wire is configured to facilitate coaxial catheter advancement over at least a part of the length of the coil wire. Existing catheters utilize the distal tip of a guidewire to select the vessel origins by shaping it (either during fabrication or by the operating physician) in such a way that the tip will point into the desired vessel as the wire is advanced. The coil wire of the present disclosure functions in a unique manner: its distal tip resumes a pre-formed three-dimensional coil configuration as it is expressed from the catheter. This shape catches the flow of moving blood and propels the coil wire forward while unfurling the coil. As the coil wire is advanced, the small outer diameter of the unfurled coil allows it to pass deep into the selected vessel.

### CROSS-REFERENCE TO RELATED APPLICATIONS

This application is a continuation-in-part application claiming priority to, and the benefit of, PCT international application no. PCT/US2021/029700, filed Apr. 28, 2021, that claims priority to, and the benefit of, U.S. provisional application No. 63/016,730 filed on Apr. 28, 2020 and U.S. provisional application No. 63/017,131 filed on Apr. 29, 2020, all of which are hereby incorporated by reference in their entireties.

### TECHNICAL FIELD

This present disclosure relates to guidewires for navigation of blood vessels.

### BACKGROUND

Catheters are used in a wide variety of medical applications for therapeutic and diagnostic purposes. For catheters that are configured for insertion into blood vessels, typically a guidewire is used to navigate into a selected blood vessel and then a catheter is advanced over the guidewire into the selected vessel. The guide wire has a distal end and a proximal end. The distal end of the guidewire is first navigated to the opening of the selected blood vessel by a user who advances the guidewire by manipulating the proximal end of the guidewire. Once the distal tip at the distal end of the guidewire has been advanced into the selected blood vessel, the catheter is advanced over the proximal end of the guidewire into the selected blood vessel.

Existing guidewires utilize the distal tip of the guidewire to select the opening of the vessel of interest by shaping the distal tip into a particular shape and/or through manipulation of the proximal end of the guidewire by the operating physician during advancement in such a way that the distal tip will point into the selected vessel as the wire is advanced. One of the problems with these approaches is that the distal tip of the distal end of the guidewire is somewhat stiff and can cause trauma to the vessel. Another problem is that it can be difficult for the distal tip to select the desired vessel due to the branching geometry or tortuosity of the vessel. Yet another problem is that high tortuosity of the vessel can lead to the tip reaching a point in the vessel from which it cannot be further advanced. In other words, a vessel having a high tortuosity can be twisted to such an extent that the distal tip becomes wedged against an inner wall of the vessel and cannot be further advanced due to reduced torque translation caused by the distal tip being wedged in the vessel.

A need exists for an improved guidewire and method that facilitate selection of the desired vessel and advancement even in cases of complex branching geometry and/or tortuosity.

### SUMMARY

The present disclosure describes a coil wire for navigation of blood vessels of a body of a patient, and a method for using the coil wire. The coil wire comprises a proximal portion, a middle portion and a distal portion. The proximal portion has proximal and distal ends and is tapered such that the proximal end has a larger diameter than the distal end. The middle portion has proximal and distal ends and is tapered such that the proximal end of the middle portion has a diameter that is substantially equal to the diameter of the distal end of the proximal portion and the distal end of the middle portion has a diameter that is smaller than the diameter of the proximal end of the middle portion. The distal portion has proximal and distal ends. The distal end of the proximal portion is coupled to the proximal end of the middle portion, and the distal end of the middle portion is coupled to the proximal end of the distal portion. The distal portion comprises a coil having a proximal end and a distal end. The coil is configured to be flow-directed such that if blood flow of an intended blood vessel exerts a sufficient force on the coil, the force exerted on the coil by the blood flow causes the coil to unfurl from a furled state into an unfurled state and to be directed in a direction of the blood flow of the intended blood vessel.

In addition to its utility in this regard, the coil wire can also be employed to retrieve biological material from the vascular environment. As the coil wire traverses blood vessels, it contacts a vessel wall and/or luminal contents. Some of this biological material becomes adherent to the coil wire and can be retrieved from the body when the coil wire is removed. Detachable embolization coils pose risk of detaching within the blood vessel and are not designed with this specific use in mind. The coil wire has been designed specifically to prevent untoward detachment in the body and is therefore suited for the purpose of endovascular biopsy.

In accordance with a representative embodiment, the coil wire is configured to allow a catheter to be advanced over the coil wire into a position in the intended blood vessel when the coil is in the unfurled state and to allow the coil wire to be withdrawn from the intended blood vessel of the patient through the catheter after the catheter is in said position.

In accordance with a representative embodiment, the proximal portion, the middle portion and the distal portion are integrally formed as a single piece part.

In accordance with a representative embodiment, the proximal portion and the middle portion are integrally formed as a single piece part, and the distal portion is formed as a piece part that is separate from the proximal and middle portions. In accordance with this representative embodiment, the coil wire further comprises an attachment mechanism disposed at a transition region where the distal end of the middle portion couples to the proximal end of the distal portion. The attachment mechanism couples the distal end of the middle portion to the proximal end of the distal portion.

In accordance with a representative embodiment, the coil wire is tapered such the coil wire has a first diameter at the proximal end of the proximal portion that is larger than a second diameter of the coil wire at the distal end of the middle portion and has a continuously decreasing diameter over a length of the coil wire extending at least from the proximal end of the proximal portion to the distal end of the middle portion.

In accordance with a representative embodiment, the coil wire is made of a metallic material to provide the coil wire with radio opacity.

In accordance with a representative embodiment, the coil wire comprises Nickel-Titanium (Nitinol).

In accordance with a representative embodiment, the coil wire comprises steel.

In accordance with a representative embodiment, the coil wire comprises cobalt-chromium.

In accordance with a representative embodiment, the proximal and middle portions comprise a first metallic material and the coil comprises a second metallic material.

In accordance with a representative embodiment, the first metallic material is selected from the group comprising Nitinol, steel and cobalt-chromium and the second metallic material comprises platinum.

In accordance with a representative embodiment, the coil of the distal portion possesses both shape memory and shape-ability. The shape-ability allows the coil to unfurl from the furled state into the unfurled state when the force of blood flow exerted on the coil is sufficient to cause the coil to unfurl from the furled state into the unfurled state. The shape memory causes the coil to attempt to return to the furled state from the unfurled state when the force of the blood flow exerted on the coil is less than said sufficient force.

In accordance with a representative embodiment, the coil in the furled state is substantially helical in shape.

In accordance with a representative embodiment, a tip of the distal end of the coil when the coil is in the unfurled state is curved so as to be nonparallel to a center axis of the coil when the coil is in the furled state, the curved tip of the distal end of the coil being atraumatic to the selected vessel.

In accordance with a representative embodiment, the distal end of the middle portion is bendable into a bent state to allow improved control over vessel selection by a user who manipulates the proximal end of the proximal portion of the coil wire to select the intended vessel.

In accordance with a representative embodiment, the proximal and distal portions of the coil wire comprise a guidewire.

In accordance with a representative embodiment, the proximal and distal portions of the coil wire comprise a hypodermic tube (hypo-tube).

In accordance with a representative embodiment, a combined length for the proximal portion and the distal portion ranges from about 150 to about 250 centimeters (cm), a length of the coil in the furled state ranges from about 1.5 millimeters (mm) to about 2.5 mm, a length of the coil in the unfurled state ranges from about 4.0 mm to about 7.0 mm, the diameter of the proximal end of the proximal portion ranges from about 0.005 inches to about 0.025 inches, the diameter of the distal end of the proximal portion ranges from about 0.005 inches to about 0.15 inches, the diameter of the proximal end of the middle portion ranges from about 0.005 inches to about 0.15 inches, the diameter of the distal end of the middle portion ranges from about 0.010 inches to 0.15 inches, and a diameter of the distal tip of the coil is less than or equal to about 0.010 inches.

In accordance with a representative embodiment, the coil wire further comprises an attachment mechanism that couples the distal end of the middle portion to the proximal end of the distal portion. The distal portion is detachable from the middle portion by manipulating the attachment mechanism.

In accordance with a representative embodiment, the method comprises: providing a coil wire comprising proximal portion, a middle portion and a distal portion, the proximal portion having proximal and distal ends, the middle portion having proximal and distal ends and the distal portion having proximal and distal ends, the proximal portion being tapered such that the proximal end of the proximal portion has a larger diameter than the distal end of the proximal portion, the middle portion being tapered such that the proximal end of the middle portion has a diameter that is substantially equal to the diameter of the distal end of the proximal portion and the distal end of the middle portion having a diameter that is smaller than the diameter of the proximal end of the middle portion, the distal end of the proximal portion being coupled to a proximal end of the middle portion, the distal end of the middle portion being coupled to the proximal end of the distal portion, the distal portion comprising a coil having a proximal end and a distal end; the coil is configured to be flow-directed such that if blood flow of an intended blood vessel exerts a sufficient force on the coil, the force exerted on the coil by the blood flow causes the coil to unfurl from a furled state into an unfurled state and to be directed in a direction of the blood flow of the intended blood vessel; advancing the coil to a branch point where the intended blood vessel and another blood vessels branch; allowing the coil to unfurl into the unfurled state as directed by the blood flow such that the distal end of the coil passes within the intended vessel to a desired location within the intended blood vessel; advancing a catheter over the coil wire into the intended blood vessel; and removing the coil wire from the body of the patient and from the catheter.

These and other features and advantages will become apparent from the following description, drawings and claims.

### BRIEF DESCRIPTION OF THE DRAWINGS

Many aspects of the invention can be better understood with reference to the following drawings. The components in the drawings are not drawn to scale, emphasis instead being placed upon clearly illustrating the principles of the present invention. Moreover, in the drawings, like reference numerals designate corresponding parts throughout the several views.

FIG. 1 illustrates a side plan view of the coil wire in accordance with a representative embodiment having a proximal portion, a middle portion and a distal portion, with the distal portion having a coiled distal end that is configured to be flow-directed.

FIG. 2 illustrates a side view of the portion of the coil wire shown in dashed box 111 in FIG. 1 in accordance with a representative embodiment in which the proximal portion, the middle portion and the distal portion are integrally formed as a single piece part.

FIG. 3 illustrates a side view of the portion of the coil wire shown in dashed box 111 in FIG. 1 in accordance with a representative embodiment in which the proximal portion and the middle portion are integrally formed as a single piece part, but the distal portion that includes the coiled distal end is a separate part that is attached to the middle portion.

FIG. 4 illustrates a side plan view of the middle and distal portions of the coil wire shown in FIGS. 1-3 with the coil of the distal portion elongated and flow-directed by the flow of blood toward and into the vessel of interest.

FIG. 5 is a flow diagram depicting the method of using the coil wire shown in FIG. 1 for navigating blood vessels of a body of a patient.

### DETAILED DESCRIPTION

The present disclosure discloses a coil wire and method of using the coil wire to navigate blood vessels that overcome the problems of the aforementioned known devices and procedures. The coil wire is designed, as are existing guidewires, to facilitate coaxial catheter advancement over at least a part of the coil wire's length. However, existing catheters utilize the distal tip of the guidewire to select vessel origins by shaping it (either during fabrication or by the operating physician) in such a way that the tip will point into the desired vessel as the wire is advanced. The coil wire of the present disclosure functions in a unique manner: its distal tip resumes a pre-formed three-dimensional coil configuration as it is expressed from the catheter. This shape catches the flow of moving blood, much like a sail catches the wind, and propels the coil wire forward while unfurling the coil. As the coil wire is advanced in the presence of flowing blood, the small outer diameter of the unfurled coil allows it to pass deep into the vessel of interest. At this point, a coaxial catheter be advanced over the coil wire into the desired vessel.

In the following detailed description, for purposes of explanation and not limitation, exemplary, or representative, embodiments disclosing specific details are set forth in order to provide a thorough understanding of the inventive principles and concepts. However, it will be apparent to one of ordinary skill in the art having the benefit of the present disclosure that other embodiments according to the present teachings that are not explicitly described or shown herein are within the scope of the appended claims. Moreover, descriptions of well-known apparatuses and methods may be omitted so as not to obscure the description of the exemplary embodiments. Such methods and apparatuses are clearly within the scope of the present teachings, as will be understood by those of skill in the art. It should also be understood that the word “example,” as used herein, is intended to be non-exclusionary and non-limiting in nature.

The terminology used herein is for purposes of describing particular embodiments only, and is not intended to be limiting. The defined terms are in addition to the technical, scientific, or ordinary meanings of the defined terms as commonly understood and accepted in the relevant context.

The terms “a,” “an” and “the” include both singular and plural referents, unless the context clearly dictates otherwise. Thus, for example, “a device” includes one device and plural devices. The terms “substantial” or “substantially” mean to within acceptable limits or degrees acceptable to those of skill in the art. For example, the term “substantially parallel to” means that a structure or device may not be made perfectly parallel to some other structure or device due to tolerances or imperfections in the process by which the structures or devices are made. The term “approximately” means to within an acceptable limit or amount to one of ordinary skill in the art. Relative terms, such as “over,” “above,” “below,” “top,” “bottom,” “upper” and “lower” may be used to describe the various elements' relationships to one another, as illustrated in the accompanying drawings. These relative terms are intended to encompass different orientations of the device and/or elements in addition to the orientation depicted in the drawings. For example, if the device were inverted with respect to the view in the drawings, an element described as “above” another element, for example, would now be below that element.

Relative terms may be used to describe the various elements' relationships to one another, as illustrated in the accompanying drawings. These relative terms are intended to encompass different orientations of the device and/or elements in addition to the orientation depicted in the drawings.

The term “coil wire,” as that term is used herein, can denote a microwire having a solid core as well as a hollow core (hypotube).

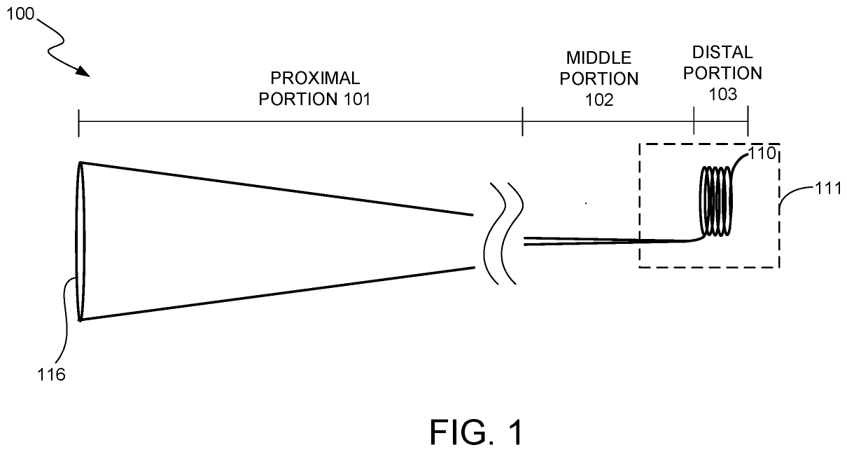

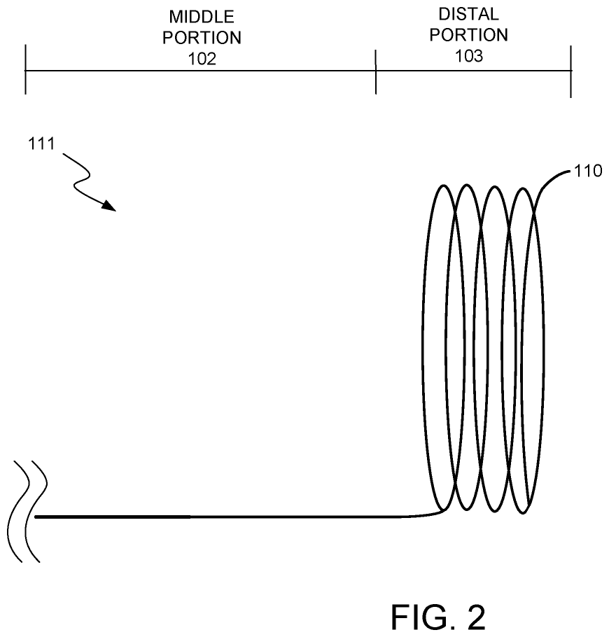

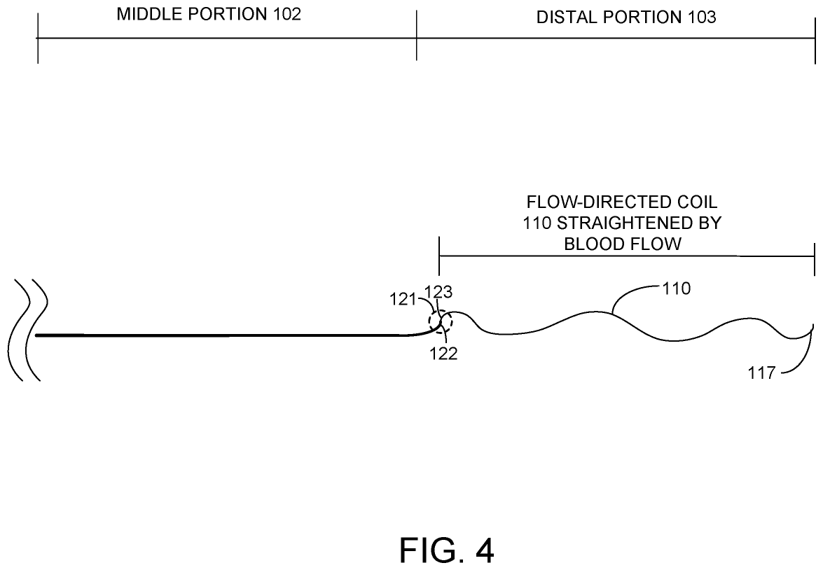

FIG. 1 illustrates a side plan view of the coil wire 100 in accordance with a representative embodiment having a proximal portion 101, a middle portion 102 and a distal portion 103. The distal portion 103 comprises a coil 110 that is configured to be flow-directed. In other words, the flow of blood into the vessel of interest causes the coil 110 to unfurl, or elongate, and to continue to track into the vessel due to the coil 110 being flow-directed. FIG. 2 illustrates a side view of the distal portion 103 of the coil wire 100 shown in dashed box 111 of FIG. 1 in accordance with a representative embodiment in which the proximal portion 101, the mid-portion 102 and the distal portion 103 are integrally formed as a single piece part. FIG. 4 illustrates a side plan view of the middle and distal portions 102 and 103, respectively, of the coil wire 100 with the coil 110 of the distal portion 103 elongated and flow-directed by the flow of blood toward and into the vessel of interest.

The coil wire 100 is typically tapered such that it is larger in diameter at the proximal end 116 of the proximal portion 101 and smaller in diameter at the location where the middle portion 102 meets the distal portion 103. Making the proximal portion 101 near the proximal end 116 larger in diameter increases the amount of torque that can be applied to the coil wire 100 at the distal end 122 of the middle portion of 102. Because the torque force increases with diameter, it is known to make the proximal end of guidewires and hypotubes as large in diameter as possible and to taper them to a smaller diameter at the distal end. It should be noted, however, that the coil wire 100 is not limited to having any particular shape or dimensions.

The coil wire 100 is typically made of a metallic material to provide it with radio opaque characteristics such that the coil wire 100 is visible during X-ray procedures. In the representative embodiment shown in FIGS. 1 and 2 in which the proximal portion 101, the middle portion 102 and the distal portion 103 are manufactured as an integrally-formed, single piece part, the coil wire 100 can be made of, for example, Nickel-Titanium (Nitinol) or a similar metallic material such as, for example, steel or Cobalt-Chromium, all of which possess both shape memory and shape-ability.

In its unfurled state shown in FIGS. 1 and 2, the coil 110 preferably has a helical shape, although other shapes are possible. In its unfurled state, the coil 110 acts as a sail that catches the blood flow in a manner similar to the manner in which a sail of a sailboat catches the wind. The coil 110 is small enough in diameter and soft enough that the flow of blood against it causes it to elongate or unfurl into a shape that can be similar to the shape shown in FIG. 4, although the shape of the coil 110 in the unfurled state can vary based on a number of factors that can depend on the use application. The blood flow directs the distal portion 103 forward, i.e., in the direction of the blood flow. This feature helps select the vessel of interest and, once the distal portion 103 is inside of the vessel of interest, helps advance the distal tip 117 (FIG. 4) of the coil 110 forward within the vessel. This overcomes the aforementioned problems of the known approaches and known guidewire structures commonly used for this purpose.

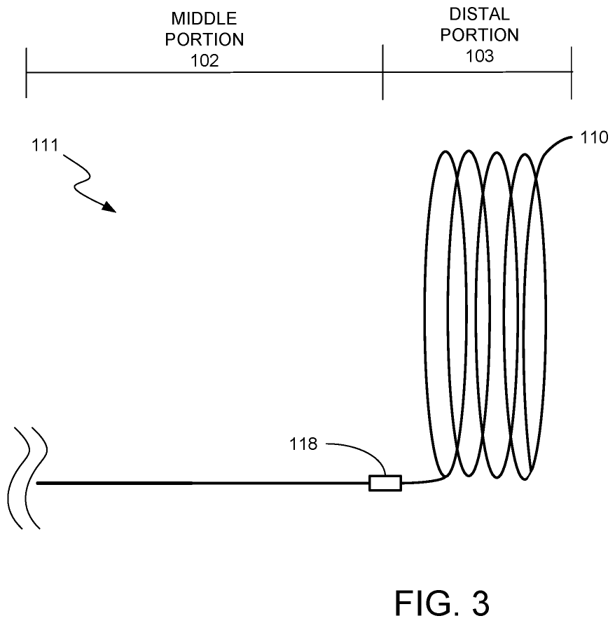

FIG. 3 illustrates a side view of the portion of the coil wire 100 shown in dashed box 111 in FIG. 1 in accordance with a representative embodiment in which the proximal portion 101 and the middle portion 102 are integrally formed as a single piece part, but the distal portion 103 that includes the coil 110 is a separate piece part that is attached to the middle portion 102 via an attachment mechanism 118. In accordance with this representative embodiment the proximal and middle portions 101 and 102, respectively, comprise a first metallic material and the coil 110 comprises a second metallic material. Any suitable metallic materials can be used for the first and second metal materials. In accordance with an embodiment, the first metallic material is selected from the group comprising Nitinol, steel and cobalt-chromium and the second metallic material comprises platinum.

Detachable coils are known in the art that are attached to a stiff pusher wire and are detachable from the pusher wire. In cases where the detachable coil is attached to the pusher wire via solder, detachment is achieved by using an electrolytic charge to burn the solder that is used to attach the coil to the pusher wire. In cases where the detachable coil is connected to the pusher wire by a locking mechanism, detachment is achieved by unlocking the locking mechanism. However, known detachable coils are generally not well suited for the goals of the coil wire 100 described herein for multiple reasons. First, the known detachable coils are meant to be detached, and therefore the transition zone between the pusher wire and the coil is very soft to facilitate detachment. The soft distal portion of the pusher wire does not facilitate coaxial advancement of a catheter. Second, it would be very difficult, and likely dangerous, to advance a catheter over the known pusher wire/detachable coil configuration because that would require pushing all of the coil out of the catheter and pushing at least some of the proximal portion of the stiff pusher wire out of the catheter and into the vessel in order to provide support to coaxially advance a catheter. The stiff pusher wire is not designed to be used in this manner. Third, because detachable coils are designed to be detached in the body of the patient, coaxially advancing a catheter over a detachable coil's detachment zone or repeatedly manipulating a detachable coil in a blood vessel may cause inadvertent detachment of the coil, which is not desirable for navigating through blood vessels.

For these reasons, such pusher wire/detachable coil configurations would not be well suited to meet the goals discussed herein. In contrast to the known pusher wire/detachable coil configurations, because the proximal and middle portions 101 and 102, respectively, of the coil wire 100 are tapered, a catheter can easily be advanced over the coil wire 100. Also, the tapered configuration makes the distal end 122 of the middle portion more flexible, shapeable and atraumatic to vessels. All of these features make the coil wire 100 well suited for navigation of vessels.

The attachment mechanism 118 can be any suitable attachment mechanism, but it should be sufficiently robust such that passing a catheter over the attachment mechanism 118 will not cause the distal portion 103 to detach from the middle portion. A variety of attachment mechanisms are suitable for bonding the middle and distal portions 102 and 103, respectively, together, as will be understood by those of skill in the art in view of the discussion provided in the present disclosure.

In some cases, however, it may be desirable to make the coil 110 detachable from the middle portion 102. In such cases, the attachment mechanism 118 can be one of the known attachment mechanisms discussed above that are used with known detachable coils (e.g., solder, locking mechanism) or some other type of attachment mechanism. Making the coil 110 detachable from the middle portion 102 can be useful in cases in which the coil 110 is meant to remain in the patient's body, such as, for example, in a blood vessel to block blood flow. For example, the coil wire 100 can be used to install several of the coils 100 in a blood vessel to block it. Once detached from the middle portion 102, the coils 110 would remain in their unfurled states inside of the vessel to block it.

With reference again to FIG. 4, another benefit of the coil wire 100 is that it can be designed and manufactured such that when the coil 110 is in its unfurled state, the distal tip 117 is slightly curved rather than pointed so that the distal tip 117 is atraumatic to the selected vessel. In addition, in the transition region 121 where the distal end 122 of the middle portion 102 meets the proximal end 123 of the coil 110, the distal end 122 of the middle portion 102 can be shaped. For example, in the field, a mandrel could be used along with a finger of the user to shape the distal end 122 into a suitable shape, such as a J shape, for example. Imparting such a shape to the distal end 122 would allow more control over vessel selection by the user or machine manipulating the proximal end 116 of the proximal portion 101.

The coil wire 100, all of it or portions of it, can be coated with a hydrophilic coating to allow the catheter to slide over it with less friction. A variety of hydrophilic coatings can be used for this purpose, including, but not limited to, a hydrophilic coating that is formed by a process in which the coil wire 100 or portions of it are placed in a gaseous atmosphere of silicone to coat the coil wire.

The coil wire 100 is not limited to having any particular dimensions, shape or configuration, other than that which are needed to achieve the goals discussed herein. An example of suitable dimension can be, for example, a combined length for the proximal portion 101 and the distal portion 102 ranging from about 150 to about 250 centimeters (cm), a length of the coil 110 in the furled state ranging from about 1.5 millimeters (mm) to about 2.5 mm, a length of the coil 110 in the unfurled state ranging from about 4.0 mm to about 7.0 mm, the diameter of the proximal end 116 of the proximal portion 101 ranging from about 0.005 inches to about 0.025 inches, the diameter of the distal end of the proximal portion ranging from about 0.005 inches to about 0.15 inches, the diameter of the proximal end of the middle portion ranging from about 0.005 inches to about 0.15 inches, the diameter of the distal end 122 of the middle portion 102 ranging from about 0.010 inches to about 0.15 inches, and a diameter of the distal tip 117 of the coil 110 being less than or equal to about 0.015 inches.

In the case in which the coil wire 100 comprises a guidewire, once the distal tip 117 of the unfurled coil 110 is at a desired location in the selected vessel, a catheter (not shown) can be advanced over the coil wire 100 and into the vessel. The coil wire 100 can then be removed from the patient, leaving the catheter in position at the desired location. For vessel selection, the user manipulates the proximal end 116 of the proximal portion 101 to advance the distal tip 117 toward the opening of the intended vessel. As the coil 110 approaches the intended vessel, the force of the blood flow exerted on the 110 causes the coil 110 to deflect as show in FIG. 4 and to be carried by the blood flow into the opening of the blood vessel as the user advances the coil wire 100.

Once the coil 110 has been pushed out of the catheter and into the blood vessel, it will unfurl due to the force of moving blood. At vessel branching points, the coil 110 with deflect or be carried into a particular branch and then deep into that branch, despite its tortuosity, because the blood flow is carrying it forward.

In terms of navigation, there are generally two maneuvers involved in using a guidewire. The same maneuvers apply to the coil wire 100 of the present disclosure. The first maneuver is to select one of two vessels at a branch point to which the coil 110 is advanced. At this branch point, or bifurcation, the coil 110 is allowed to “flicker” in the blood flow until it “flicks” into the desired branch. Because the coil 110 is flow directed, it tends to select the branch with higher blood flow, which is desirable in vessel navigation. If difficulty is encountered at this step, the distal end 122 (FIG. 4) of the middle portion 102 can be shaped by the operator into a gentle curve so that the coil is presented to the orifice of the desired branch. Second, once the coil 110 has selected the desired branch, the proximal end 116 (FIG. 1) is pushed into the catheter hub. Again, because the coil 110 is flow directed it will not be hindered by vessel tortuosity and become stuck along a vessel wall. If the coil 110 begins to furl or bunch up, the operator stops pushing and allows the coil 110 to unfurl again. Barring this, the coil wire 100 can be retracted slightly to achieve coil unfurling. Then, the coil wire is again pushed forward by the operator.

The coil wire can also be employed to retrieve biological material from the vascular environment. The coil wire can be advanced forwards and retracted backwards within the blood vessel which is intended to undergo biopsy. Relevant cell types include all those composing the blood vessel and circulating through in the blood such as, but not limited to, endothelial cells, smooth muscle cells, fibroblasts, tumor/cancer cells, red blood cells and white blood cells. As the coil wire is advanced and retracted the soft distal tip (coil portion) will contact the vessel wall and may even curl up in a rounded configuration within the vessel, contacting the vessel walls at several locations along the coil. After a period of time, which can extend between several seconds and a few minutes, the coil is retracted back into the catheter (e.g., a microcatheter) and completely removed from the body. Additionally, an electrical charge may be applied to the coil wire at the proximal end in such a way as to transiently induce a change in electrical potential at the distal (coil) end. This charge may also be used to promote adherence of cells to the coil wire. Cells from the biopsied blood and/or blood vessel adherent to the coil can then be separated from the coil wire using chemical means such as, e.g., cell dissociation buffer and/or mechanical methods such as, e.g., vortex separation. Using this method of endovascular biopsy, single cells ranging in number from a few (1's) to several (10's) may be retrieved from the blood vessel using the coil wire. Unlike detachable coils currently in use, the coil wire is not detachable and therefore specifically suited to endovascular biopsy as there is no risk of the coil detaching from the coil wire, even following maneuvers such as advancing and retracting the coil or applying an electrical current, both of which are known to increase the chances of a conventional embolization coil separating from the proximal pusher wire. The coil wire is designed as a non-detachable device for use in vascular navigation and biopsy as opposed to detachable coils which are designed as embolic agents.

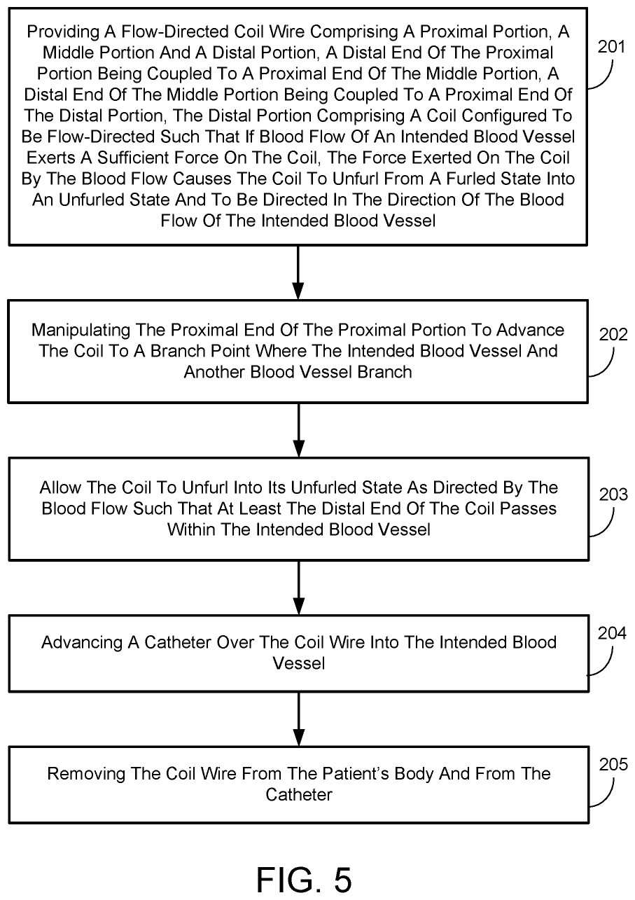

FIG. 5 is a flow diagram depicting the method of using the coil wire 100 for navigating blood vessels of a body of a patient. Bock 201 represents the step of providing the coil wire 100 described above. As indicated above, the coil of the coil wire is configured to be flow-directed such that if blood flow of an intended blood vessel exerts a sufficient force on the coil, the force exerted on the coil by the blood flow causes the coil to unfurl from a furled state into an unfurled state and to be directed in a direction of the blood flow of the intended blood vessel. Block 202 represents the step of a user or machine (hereinafter referred to as “the operator”) manipulating the proximal end of the proximal portion to advance the coil to a branch point where the intended blood vessel and another blood vessel branch. Block 203 represents the step of allowing the coil to unfurl into the unfurled state as directed by the blood flow such that the distal end of the coil passes within the intended vessel to a desired location within the intended blood vessel. Block 204 represents the step of the operator advancing a catheter over the coil wire into the intended blood vessel. In some cases, the coil wire can be manipulated (e.g., advanced forwards and retracted backwards) within the blood vessel which is intended to undergo biopsy as previously described. Block 205 represents the step of removing the coil wire from the body of the patient and from the catheter. The coil can be retracted back into the catheter and completely removed from the body. Cells from the biopsied blood and/or blood vessel adherent to the coil can then be separated from the coil wire for further analysis.

It should be emphasized that the above-described embodiments are examples of the manner in which the coil wire 100 can be configured and implemented and are set forth to provide a clear understanding of the inventive principles and concepts. Many variations and modifications can be made to the above-described embodiments of the coil wire 100 without departing from the scope of the present disclosure, as will be understood by those of skill in the art in view of the description provided herein. All such modifications and variations are intended to be included herein within the scope of this disclosure and the present invention.

What is claimed is:

1. A coil wire for navigation of blood vessels of a body of a patient, the coil wire comprising:
a proximal portion, a middle portion and a distal portion, the proximal portion having proximal and distal ends and being tapered such that the proximal end has a larger diameter than the distal end, the middle portion having proximal and distal ends and being tapered such that the proximal end of the middle portion has a diameter at that is substantially equal to the diameter of the distal end of the proximal portion, the distal end of the middle portion having a diameter that is smaller than the diameter of the proximal end of the middle portion, the distal portion having proximal and distal ends, the distal end of the proximal portion being coupled to the proximal end of the middle portion, the distal end of the middle portion being coupled to the proximal end of the distal portion, the distal portion comprising a coil having a proximal end and a distal end, the coil being configured to be flow-directed such that if blood flow of an intended blood vessel exerts a sufficient force on the coil, the force exerted on the coil by the blood flow causes the coil to unfurl from a furled state into an unfurled state and to be directed in a direction of the blood flow of the intended blood vessel.

2. The coil wire of claim 1, wherein the coil wire is configured to allow a catheter to be advanced over the coil wire into a position in the intended blood vessel when the coil is in the unfurled state and to allow the coil wire to be withdrawn from the intended blood vessel of the patient through the catheter after the catheter is in said position.

3. The coil wire of claim 1, wherein the proximal portion, the middle portion and the distal portion are integrally formed as a single piece part.

4. The coil wire of claim 1, wherein the proximal portion and the middle portion are integrally formed as a single piece part, and wherein the distal portion is formed as a piece part that is separate from the proximal and middle portions, the coil wire further comprising:
an attachment mechanism disposed at a transition region where the distal end of the middle portion couples to the proximal end of the distal portion, the attachment mechanism coupling the distal end of the middle portion to the proximal end of the distal portion.

5. The coil wire of claim 1, wherein the coil wire is tapered such the coil wire has a first diameter at the proximal end of the proximal portion that is larger than a second diameter of the coil wire at the distal end of the middle portion and has a continuously decreasing diameter over a length of the coil wire extending at least from the proximal end of the proximal portion to the distal end of the middle portion.

6. The coil wire of claim 5, wherein the coil wire is made of a metallic material to provide the coil wire with radio opacity.

7. The coil wire of claim 6, wherein the coil wire comprises Nickel-Titanium (Nitinol).

8. The coil wire of claim 6, wherein the coil wire comprises steel.

9. The coil wire of claim 6, wherein the coil wire comprises cobalt-chromium.

10. The coil wire of claim 6, wherein the proximal and middle portions comprise a first metallic material and the coil comprises a second metallic material.

11. The coil wire of claim 10, wherein the first metallic material is selected from the group comprising Nitinol, steel and cobalt-chromium and the second metallic material comprises platinum.

12. The coil wire of claim 1, wherein the coil of the distal portion possesses both shape memory and shape-ability, the shape-ability allowing the coil to unfurl from the furled state into the unfurled state when the force of blood flow exerted on the coil is sufficient to cause the coil to unfurl from the furled state into the unfurled state, the shape memory causing the coil to attempt to return to the furled state from the unfurled state when the force of the blood flow exerted on the coil is less than said sufficient force.

13. The coil wire of claim 12, wherein the coil in the furled state is substantially helical in shape.

14. The coil wire of claim 12, wherein a tip of the distal end of the coil when the coil is in the unfurled state is curved so as to be nonparallel to a center axis of the coil when the coil is in the furled state, the curved tip of the distal end of the coil being atraumatic to the selected vessel.

15. The coil wire of claim 12, wherein the distal end of the middle portion is bendable into a bent state to allow improved control over vessel selection by a user who manipulates the proximal end of the proximal portion of the coil wire to select the intended vessel.

16. The coil wire of claim 1, wherein the proximal and distal portions of the coil wire comprise a guidewire.

17. The coil wire of claim 1, wherein the proximal and distal portions of the coil wire comprise a hypodermic tube (hypo-tube).

18. The coil wire of claim 1, wherein a combined length for the proximal portion and the distal portion ranges from about 150 to about 250 centimeters (cm), a length of the coil in the furled state ranging from about 1.5 millimeters (mm) to about 2.5 mm, a length of the coil in the unfurled state ranging from about 4.0 mm to about 7.0 mm, the diameter of the proximal end of the proximal portion ranging from about 0.005 inches to about 0.025 inches, the diameter of the distal end of the proximal portion ranging from about 0.005 inches to about 0.15 inches, the diameter of the proximal end of the middle portion ranging from about 0.005 inches to about 0.15 inches, the diameter of the distal end of the middle portion ranging from about 0.010 inches to 0.15 inches, and a diameter of the distal tip of the coil being less than or equal to about 0.010 inches.

19. The coil wire of claim 1, further comprising an attachment mechanism that couples the distal end of the middle portion to the proximal end of the distal portion, wherein the distal portion is detachable from the middle portion by manipulating the attachment mechanism.

20. A method for navigating blood vessels of a body of a patient, the method comprising:
providing a coil wire comprising proximal portion, a middle portion and a distal portion, the proximal portion having proximal and distal ends, the middle portion having proximal and distal ends and the distal portion having proximal and distal ends, the distal end of the proximal portion being coupled to a proximal end of the middle portion, the distal end of the middle portion being coupled to the proximal end of the distal portion, the distal portion comprising a coil having a proximal end and a distal end, the coil being configured to be flow-directed such that if blood flow of an intended blood vessel exerts a sufficient force on the coil, the force exerted on the coil by the blood flow causes the coil to unfurl from a furled state into an unfurled state and to be directed in a direction of the blood flow of the intended blood vessel;
advancing the coil to a branch point where the intended blood vessel and another blood vessels branch;
allowing the coil to unfurl into the unfurled state as directed by the blood flow such that the distal end of the coil passes within the intended vessel to a desired location within the intended blood vessel;
advancing a catheter over the coil wire into the intended blood vessel; and
removing the coil wire from the body of the patient and from the catheter.

21. The method of claim 20, comprising manipulating the coil within the intended blood vessel to obtain biopsy cells.

22. The method of claim 21, comprising separating the biopsy cells from the coil.
